(19) United States
(12) Patent Application Publication
(10) Pub. No.: US 2008/0053453 A1
(43) Pub. Date: Mar. 6, 2008

(54) ENDOTRACHEAL TUBE PLACEMENT TOOL

(76) Inventor: Mark A. Lauer, St. Paul, MN (US)

Correspondence Address: NAWROCKI, ROONEY & SIVERTSON;SUITE 401, BROADWAY PLACE EAST, 3433 BROADWAY STREET NORTHEAST, MINNEAPOLIS, MN, 554133009, US

(21) Appl. No.: 11/470,477

(22) Filed: Sep. 6, 2006

(51) Int. Cl. A61B 17/00; A62B 9/06; A61M 16/00

(52) U.S. Cl. 128/207.14; 606/1

(57) ABSTRACT

A tool for assisting a medical professional in inserting an endotracheal tube in the trachea of a patient comprises a relatively rigid rod in the shape of an offset spiral. The tool has a mandrel section, a curved arc section smoothly transitioning from the mandrel section, and a tip section smoothly transitioning from the arc section. The arc section is an arc of a three dimensional spiral, and angularly and spatially offsets the tip section with respect to the mandrel section.

### BACKGROUND OF THE INVENTION

An endotracheal (e/t) tube is a medical device in the form of a tube that is inserted to extend through a patient's glottic opening and into the trachea to assist breathing. An e/t tube for an adult patient typically comprises a soft, pliable plastic hose, perhaps 10-11.5 mm. OD and 6.5-8.0 mm. ID with a uniform cross section, and with a length typically ranging from 30-35 cm. A pediatric e/t tube may be approximately two-thirds the length and OD of an adult e/t tube. An infant e/t tube may be one-half the size of an adult e/t tube. E/t tubes must be pliable so that they can conform to the shape of the patient's throat, and so they can bend within the mouth to protrude from the patient's lips.

An e/t tube has a leading end that a medical professional inserts in the patient's throat and pushes into the trachea to a distance of 2-4 cm. The length of the e/t tube allows a trailing end of an inserted e/t tube to project from the patient's mouth.

To keep the e/t tube in place, an e/t tube typically has near the leading end and surrounding the outer surface, a balloon-like cuff that is blown up once the e/t tube is inserted into the trachea. A small tube that passes through the wall of the e/t tube between the leading and trailing ends and extends from the patient's mouth, provides a means for inflating the balloon. A trailing end of the e/t tube has a fitting for attachment to an air supply.

When a medical professional inserts an e/t tube in a patient's trachea to provide airflow through a closed or restricted breathing passage, often the patient is already unconscious. The e/t tube must rapidly and accurately enter the trachea during placement, since time is precious when breathing is difficult for a person. At the same time, inserting the e/t tube should not cause scraping or tearing of the soft throat tissue.

It is difficult for a medical professional to consistently place the e/t tube in the trachea. The flexibility of typical e/t tubes sometimes makes it difficult to insert the e/t tube in a trachea that may already be somewhat constricted. Also, the esophageal opening is very close to the glottal opening to the trachea, sometimes causing the e/t tube to enter the esophagus instead of the trachea. For all of these reasons, personnel cannot always place an e/t tube in a trachea as quickly and accurately as is desirable. This is caused by some patients' anatomy. The professional can usually detect improper e/t tube placement, but the delay is undesirable.

Tools currently exist for assisting e/t tube placement. Laryngoscopes are one type of such tools. These provide for viewing the glottic opening at the entrance to the trachea during e/t tube placement, using either an optical system or a video camera. They do allow placement accuracy around 90-95%.

An endobronchialscope (e/b scope) may be a better alternative than a laryngoscope for intubating patients with more difficult anatomy. An e/b scope is a long, thin, flexible tube with a video camera at the tip. The e/b scope is thin enough to be inserted completely through the e/t tube duct from the trailing end to the leading end prior to starting the placement. The video camera in the e/b scope tip allows a good view of the glottic opening. Once the e/b scope tip is properly positioned, the e/t tube is pushed off the e/b scope and into the trachea. The inventor estimates that an e/b scope allows 96-98% e/t tube placement accuracy. E/b scopes cost thousands of dollars, and hence are not disposable devices. Therefore, they require sterilization between uses, which may some times be inconvenient.

For all of these reasons, a better tool for inserting e/t tubes in patients' throats is desirable.

### BRIEF DESCRIPTION OF THE INVENTION

A tool in the nature of a stylet temporarily placed into the duct of an e/t tube is an effective means for assisting in rapidly and accurately inserting an e/t tube in the throat of a living being. Such a tool comprises a relatively rigid rod in a general shape referred to hereafter as an offset spiral. The tool has mandrel and handle sections that a medical professional holds during the insertion procedure. A curved arc section of the tool has a generally spiral configuration and smoothly transitions from the mandrel section. A tip section of the tool smoothly transitions from the arc section. The tip section may be curved or may also be nearly straight. The arc section angularly offsets the tip section with respect to the mandrel section. Preferably, the mandrel, arc, and tip sections are unitary, and are formed from a single piece of rod or wire.

The term “offset spiral” in this context means that the curved arc section does not lie in a plane.

The term “relatively rigid” means that the tool comprises a rod formed from material that is stiff enough to allow a skilled medical professional to properly place the e/t tube at the glottic opening of a patient without permanently bending the tool from the specified shape. It is convenient to form the stylet tool from heavy gauge, relatively straight, metal wire or rod by bending into the specified shape, which inherently makes the tool relatively rigid.

The term “angularly offset” means that a portion of the tip section adjacent to the tip is at an acute angle to a plane in which at least a part of the mandrel section lies.

The arc section is in the general shape of an arc of a three dimensional spiral. The mandrel and tip sections may also have a similar three dimensional spiral shape, or may be substantially linear.

The mandrel section may have a first end and a second end. The second end of the mandrel section is unitary with the arc section. A handle section of the tool may be attached to the mandrel section's first end.

### BRIEF DESCRIPTION OF THE DRAWINGS

FIGS. 1, 2, and 3 are respectively, top, end and side orthographic projections of an e/t tube placement tool.

### DESCRIPTION OF THE PREFERRED EMBODIMENTS

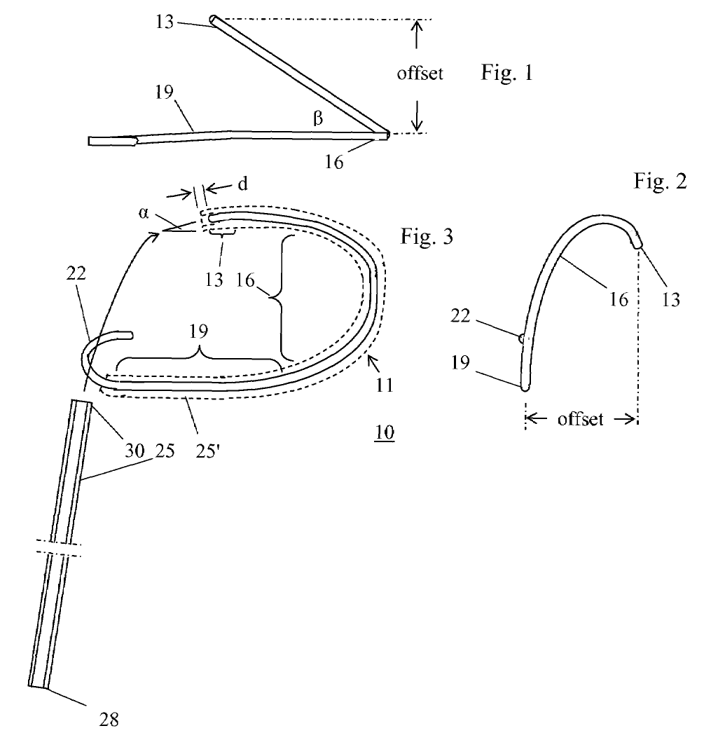

FIGS. 1, 2, and 3 collectively show an e/t tube placement tool 10 in the form of a stylet for inserting an e/t tube 25 in a patient's trachea. FIGS. 1-3 are aligned as orthographic projections of the top, end, and side of tool 10. FIG. 3 shows e/t tube 25 in phantom at 25′ mounted on tool 10 to form a tool assembly 11 that is prepared for use by a medical professional to insert e/t tube 25 in a patient's trachea. For simplicity, tube 25 is omitted from FIGS. 1 and 2. For the same reason, the balloon and attachment fitting mentioned in the Background section are omitted from e/t tube 25. E/t tube 25 has leading and trailing ends 28 and 30 respectively.

Principles of descriptive geometry teach that three orthographic projections are sufficient to completely describe the shape of a simple curved line. Tool 10 may be represented as a simple curved line. Hence, the orthographic projections of FIGS. 1-3 are sufficient to define the shape of tool 10 in a manner allowing one with skill in the art to understand and reproduce tool 10.

Tool 10 preferably is formed from a straight section of a unitary, round rod or wire with a substantially uniform diameter. This piece of rod may be from 35-40 mm. long and made of a material whose yield strength and ductility allow bending into the shape shown in FIGS. 1-3. The diameter of the rod from which tool 10 is formed may be around 2.25-2.5 mm., and surely substantially smaller than the ID of e/t tube 25, so as to allow tool 10 to easily slip into and from e/t tube 25.

Tool 10 may comprise a non-toxic, inert material such as stainless steel that is relatively stiff, but yet bendable into a shape having gentle curves using sufficient force. Tool 10 should have sufficient stiffness to resist distortion when subjected to moderate bending load but be bendable into the shape shown when subjected to a greater bending load when tool 10 is formed into the shape of FIGS. 1-3. Materials with a modulus of elasticity approximately in the range of 15-40×10⁶ psi. and a yield strength of approximately 20,000-80,000 psi, such as 300 or 400 series stainless steel are suitable for the purpose. Tool 10 may also be molded from hard plastic having suitable strength and modulus of elasticity.

One version of tool 10 suitable for use with adult patients comprises at least four distinct sections. Each of the sections smoothly transitions and merges into the adjacent section(s). Forming tool 10 from a single length of rod simplifies the making of smooth transitions and curves.

A handle section 22 may be bent in an approximate hook shape as seen in FIG. 3. A mandrel section 19 perhaps 7-10 cm. long connects handle section 22 to an arc section 16. The mandrel section 19 may be straight or nearly so.

Arc section 16 may have an average radius of curvature of from 6-12 cm. and be approximately 15-20 cm. long. Arc section 16 may subtend a total angle of 150-220°. A pediatric version of tool 10 for older children might be ⅔d the size of an adult version and scaled to the size of a pediatric e/t tube. An infant version of tool 10 for very young children might be roughly ½ the size of an adult version and scaled appropriately for use on an infant patient. A similar device for veterinary practice should have the various dimensions changed to match the physiology of the animal involved.

A tip section 13 is connected to arc section 16 and supports the leading end of e/t tube 25. The tip section 13 and the adjacent portion of arc section 16 may also be nearly straight, perhaps with a radius of curvature in the range of 10-20 cm.

An important feature of tool 10 is that arc section 16 is angularly offset from the plane of FIG. 3, as can be seen in FIG. 2. The offset results from twist in the arc section 16 along the axis thereof. FIG. 1 shows the offset as an angle β that may in practice range from 15-45°. The offset spacing or distance shown in FIGS. 1 and 2 may be on the order of 5-10 cm. The angle a at which tip section 13 is pointed may range from +15 to +45°, so that the projection of tip section 13 may make an angle of 195-225° with at least a portion of the projection of mandrel section 19.

This offset feature allows a medical professional to easily and safely insert an e/t tube 25 mounted on tool 10 into a patient's trachea. E/t tube 25 is shown mounted on tool 10 in FIG. 3 as a phantom image 25′. It is easy to mount an e/t tube 25 on tool 10 by sliding tool 10 into the duct of e/t tube 25 at the trailing end until the end of tool 10 nears the leading end of e/t tube 25, to thereby form the tool assembly 11. Preferably, the leading end of e/t tube 10 should project past the tip of tool 25 by a distance d. The value d may range from approximately 0.3 to 2.5 cm. The overall length of a tool 10 may be chosen so that an amount of tool 10 protrudes from the trailing end of e/t tube 25 sufficient to provide a handle section 22 to be held by a medical professional while placing the e/t tube in a patient's trachea.

To place or insert an e/t tube 25, the medical professional first inserts tool 10 into e/t tube 25 to form a tool assembly 11, although it may be convenient to commercially provide complete tool assemblies 11. Next, she places the leading end of e/t tube 25 in assembly 11 at the glottic opening near the entrance to a patient's throat. The topology of tool 10 as shown in FIGS. 1-3 allows the medical professional to place the handle section 22 and mandrel section 19 near the patient's right cheek and outside his or her mouth during this positioning of e/t tube 25.

Next, the professional uses handle 22 to hold tool 10 relatively stationary and slowly slides e/t tube 25 off tool 10 and into the patent's trachea. The shape of tool 10 allows the medical professional to point tip section 13 directly into the patient's trachea during placement, so that e/t tube 25 slides easily into the patient's trachea with minimal discomfort or trauma. Further, the tool 10 allows the medical professional to place the e/t tube 25′ in the patient's trachea to the proper depth almost every time on the first try. Of course, the sense of the spiral and offset in arc section 16 can be reversed so that tool 10 is deployed with the mandrel section 19 near the patient's left cheek during placement of e/t tube 25. Current standard practice is to approach patients during intubation from their right side.

The offset angle feature in arc section 16 allows the user to position the handle 22, mandrel section 19, and perhaps a small portion of the arc section 16 adjacent to the mandrel section 19, near the cheek and jaw of the patient. When the professional sees or feels that e/t tube 25 is positioned properly in the patient's trachea, the professional holds e/t tube 25 near the trailing end, and then rotates tool 10 clockwise (FIG. 3 view) to withdraw tool 10 from e/t tube 25. After tool 10 is removed from e/t tube 25, the cuff (not shown) is inflated to hold e/t tube 25 securely in the patient's trachea.

Of course, many variations of tool 10 providing similar functionality are possible.

1. A tool for assisting in inserting an endotracheal tube in the throat of a patient, comprising a relatively rigid rod in the shape of an offset spiral and having a mandrel section, a curved arc section smoothly transitioning from the mandrel section, and a tip section smoothly transitioning from the arc section, said arc section angularly offsetting the tip section with respect to the mandrel section.

2. The tool of claim 1, wherein the mandrel section has a first end and a second end, said second end connecting to the midsection, and further comprising a handle attached to the mandrel section's first end.

3. The tool of claim 2, wherein the handle comprises a portion of the rod bent into the shape of a hook.

4. The tool of claim 1, wherein the spiral formed by a projection of the tool onto a surface turns through an angle of at least 150° relative to a central portion of the mandrel.

5. The tool of claim 1, wherein the spiral formed by a projection of the tool onto a surface turns through an angle of at least 180° relative to a central portion of the mandrel.

6. The tool of claim 1, wherein the spiral formed by a projection of the tool onto a surface turns through an angle of at least 210° relative to a central portion of the mandrel.

7. The tool of claim 4, wherein a portion of the tip section is angularly offset from the mandrel section by from about 15 to 45°.

8. The tool of claim 1, wherein a portion of the tip section is angularly offset from the mandrel section by from about 15 to 45°.

9. A method for using the tool of claim 1, comprising the steps of
i) inserting the tool in the duct of an endotracheal tube having a leading end and a trailing end, from the trailing end to near the leading end of the endotracheal tube;
ii) placing the endtotracheal tube leading end in the patient's mouth at the glottic opening therein;
iii) pushing the endotracheal tube of the tool to advance the endotracheal tube's leading end into the patient's trachea; and
iv) withdrawing the tool from the endotracheal tube.

10. The method of claim 9, wherein the withdrawing step includes the step of holding the endotracheal tube near the trailing end while withdrawing the tool from the endotracheal tube.

11. The tool of claim 1, including a handle section attached to the mandrel section opposite to the connections between the mandrel and arc sections.

12. The tool of claim 11, wherein the handle section comprises a portion of the rod bent into a hook shape.

13. A medical device assembly comprising the tool of claim 1 and an endotracheal tube having a duct running the entire length, wherein the tool is within the endotracheal tube duct.

14. The device of claim 13, wherein the tool's tip section has an end, and wherein the endotracheal tube has a leading end to be inserted into the patient's trachea, wherein the tool's tip section is within the endotracheal tube duct and within approximately 0.3 to 2.5 cm of the endotracheal tube leading end.
